(19) United States
(12) Patent Application Publication
(10) Pub. No.: US 2024/0140896 A1
(43) Pub. Date: May 2, 2024

(54) PROCESS FOR THE PURIFICATION OF VANILLIN OR A VANILLIN DERIVATIVE OBTAINED BY A BIOTECHNOLOGICAL PROCESS

(71) Applicant: SPECIALTY OPERATIONS FRANCE, Lyon (FR)

(72) Inventors: Talsida Zougmore, Francheville (FR); Rachid Djillali, Lyon (FR); Laurent Garel, Lyon (FR)

(73) Assignee: SPECIALTY OPERATIONS FRANCE, Lyon (FR)

(21) Appl. No.: 18/550,941

(22) PCT Filed: Mar. 14, 2022
(86) PCT No.: PCT/EP2022/056528

§ 371 Date: Sep. 15, 2023

(30) Foreign Application Priority Data
Mar. 15, 2021 (FR) 2102535

(51) Int. Cl. C07C 45/79; C07C 45/80; C07C 45/83

(52) U.S. Cl. CPC C07C 45/79; C07C 45/80; C07C 45/83

(57) ABSTRACT

The present invention relates to a process for purifying a fermentation must (M), obtained via a biotechnological process, comprising biomass and vanillin or derivatives thereof, said purification process comprising:
(a) a step of separating the biomass of a fermentation must (M) from an aqueous stream (A1) comprising vanillin or derivatives thereof,
(b) at least one step of liquid/liquid extraction of the aqueous stream (A1) comprising vanillin or derivatives thereof, said extraction step allowing the separation of an aqueous stream (A2) and an organic stream (O1) comprising vanillin or derivatives thereof,
(c) at least one step of concentrating the organic stream (O1) to obtain an organic stream (O2) in which compounds with a boiling point lower than that of vanillin or derivatives thereof are separated from said organic stream (O1),
(d) at least one step in which compounds with a boiling point higher than that of vanillin or derivatives thereof are separated from an organic stream (O2) comprising vanillin or derivatives thereof to obtain a stream (O3),
(e) at least one step of crystallizing the vanillin or derivatives thereof contained in the stream (O3).

### FIELD OF THE INVENTION

The present invention relates to a process for purifying vanillin or a vanillin derivative obtained via a biotechnological process.

### PRIOR ART

Vanillin may be obtained by various methods known to those skilled in the art, notably by the following two routes:
a “natural” route based on a biotechnological process notably comprising the cultivation of a microorganism that is capable of enabling the biotransformation of a fermentation substrate into vanillin. Such a process, in which the fermentation substrate is ferulic acid, is notably known from patent application EP 0885968. Patent U.S. Pat. No. 5,017,388 describes a process in which the fermentation substrate is eugenol and/or isoeugenol. These processes lead to the preparation of a vanillin known as natural vanillin.
a “synthetic” route comprising conventional chemical reactions starting from guaiacol and not involving microorganisms. This process leads to the preparation of a vanillin known as synthetic vanillin.

Finally, vanillin can also be prepared via a “biobased” route, in which vanillin is derived from lignin. In particular, mention may be made of U.S. Pat. No. 2,745,796, DE 1132113 and the article entitled “Preparation of lignin from wood dust as vanillin source and comparison of different extraction methods” by Azadbakht et al. in International Journal of Biology and Biotechnology, 2004, vol. 1, No. 4, pages 535-537.

At the present time, natural vanillin can be purified via the process described in patent application EP 2791098, which includes a step of liquid/liquid extraction of impurities with a pKa higher than that of vanillin. The yield for this process is good, generally greater than 80%, but in order to obtain improved organoleptic properties such as the odor and/or color of vanillin, several additional purification steps are required, thus causing the overall yield of this process to fall. The overall energy efficiency of this process is also degraded due to the use of large amounts of solvents.

International patent application WO 2014/114590 also describes a process for purifying natural vanillin. This process consists in evaporating natural vanillin, which may be performed by distillation or by vacuum evaporation of molten vanillin. This process is capable of producing very pure natural vanillin, in good yield, with a device that is simple to use and which operates continuously so as to be compatible with industrial processes. However, such a process may be difficult to implement due to the number and size of the items of equipment required.

Moreover, vanillin or derivatives thereof obtained via a biotechnological process may contain certain impurities with boiling points very close to that of vanillin or derivatives thereof. Thus, it is necessary to size the equipment suitably so as to enable efficient separation of vanillin or derivatives thereof from these products. This usually involves extending the residence time in the distillation equipment, which may generate new impurities due to the high-temperature instability of vanillin and/or impurities.

For this reason, it would be advantageous to have an improved process, notably in terms of environmental and/or energy impact, while at the same time improving the overall purification yield and also the yield of vanillin or derivatives thereof. It is also important that the vanillin purification process allows the production of a vanillin whose organoleptic properties are preserved notably in terms of taste, color and/or odor.

### BRIEF DESCRIPTION

The present invention relates to a process for purifying a fermentation must (M), obtained via a biotechnological process, comprising biomass and vanillin or derivatives thereof, said purification process comprising:
(a) a step of separating the biomass of a fermentation must (M) from an aqueous stream (A1) comprising vanillin or derivatives thereof,
(b) at least one step of liquid/liquid extraction of the aqueous stream (A1) comprising vanillin or derivatives thereof, said extraction step allowing the separation of an aqueous stream (A2) and an organic stream (O1) comprising vanillin or derivatives thereof,
(c) at least one step of concentrating the organic stream (O1) to obtain an organic stream (O2) in which compounds with a boiling point lower than that of vanillin or derivatives thereof are separated from said organic stream (O1),
(d) at least one step in which compounds with a boiling point higher than that of vanillin or derivatives thereof are separated from an organic stream (O2) comprising vanillin or derivatives thereof to obtain a stream (O3),
(e) at least one step of crystallizing the vanillin or derivatives thereof contained in the stream (O3).

### DESCRIPTION OF THE FIGURES

FIGS. 1 and 2 schematically show various processes for purifying vanillin or derivatives thereof obtained via a biotechnological process according to the present invention.

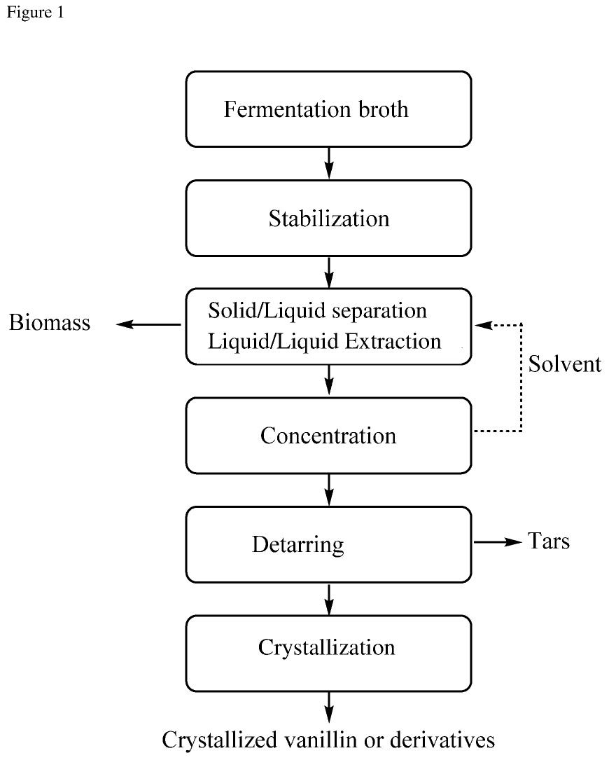

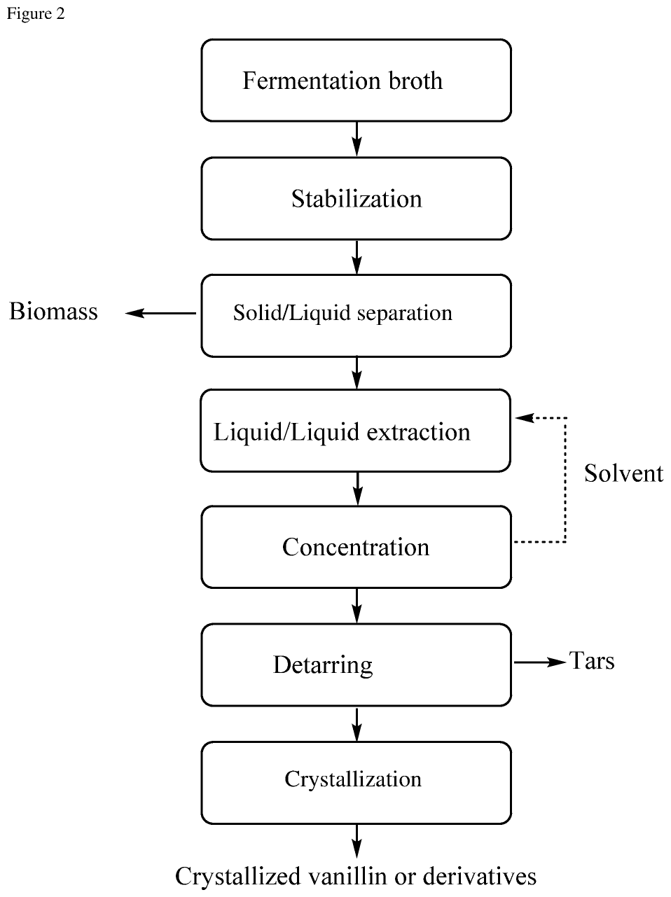

### DETAILED DESCRIPTION

In the context of the present invention, and unless otherwise indicated, the expression “between . . . and . . . ” includes the limits.

In the present invention, the term “natural vanillin” denotes a vanillin obtained via a biotechnological process. Thus, a process for preparing natural vanillin denotes herein a biotechnological process comprising the cultivation of a microorganism that is capable of enabling the transformation of a fermentation substrate into vanillin. The microorganism may be of wild-type origin or may be a genetically modified microorganism (GMM) obtained via molecular biology. Very preferentially, it may be a ferulic acid fermentation process, such as that described in patent application EP 0885968. According to a particular aspect, vanillin may be produced via a glucose or protocatechuic acid fermentation process as described in patent application WO 2013/022881.

In the context of the present invention, the term “vanillin derivative” refers to any compound that may be derived from vanillin and in particular to vanillin in salified form or glucovanillin.

In the context of the present invention, the term “vanillyl alcohol derivative” refers to any compound that may be derived from vanillyl alcohol and in particular to vanillyl alcohol in salified form or to vanillyl alcohol glucoside.

In the context of the present invention, the term “crystallization” refers to a process in which a substance passes into the solid state via a physical process, notably such as lowering the temperature.

The present invention relates to a process for purifying a fermentation must (M), obtained via a biotechnological process, comprising biomass and vanillin or derivatives thereof, said purification process comprising:
(a) a step of separating the biomass of a fermentation must (M) from an aqueous stream (A1) comprising vanillin or derivatives thereof,
(b) at least one step of liquid/liquid extraction of the aqueous stream (A1) comprising vanillin or derivatives thereof, said extraction step allowing the separation of an aqueous stream (A2) and an organic stream (O1) comprising vanillin or derivatives thereof,
(c) at least one step of concentrating the organic stream (O1) to obtain an organic stream (O2) in which compounds with a boiling point lower than that of vanillin or derivatives thereof are separated from said organic stream (O1),
(d) at least one step in which compounds with a boiling point higher than that of vanillin or derivatives thereof are separated from an organic stream (O2) comprising vanillin or derivatives thereof to obtain a stream (O3),
(e) at least one step of crystallizing the vanillin or derivatives thereof contained in the stream (O3).

In the context of the present invention, on conclusion of a biotechnological process for the preparation of vanillin or derivatives thereof, the vanillin or derivatives thereof are in aqueous solution in a fermentation must (M). Thus, the process of the present invention consists in purifying a fermentation must (M) comprising vanillin or derivatives thereof. In general, the concentration of vanillin or derivatives thereof in the fermentation must (M) is between 0.2% and 5% by weight, preferably between 0.8% and 2.5% by weight, very preferentially between 1.0% and 1.8% by weight. The aqueous solution also comprises biomass: in general, the amount of biomass is between 0.5% and 5% by weight of dry matter, preferably between 1.0% and 2.0% by weight of dry matter. The fermentation must (M) may also comprise other compounds such as ferulic acid and derivatives thereof, vanillic acid, vanillyl alcohol or derivatives thereof, coumaric acid, para-hydroxybenzaldehyde, or guaiacol. These compounds may be separated from the vanillin or derivatives thereof via the process of the present invention. Advantageously, at least one compound chosen from ferulic acid, ferulic acid derivatives, vanillic acid, vanillyl alcohol or derivatives thereof, coumaric acid, para-hydroxybenzaldehyde or guaiacol may be upgraded notably as a synthetic intermediate.

Step (a)

The purification process of the present invention comprises a step (a) of separating the biomass of a fermentation must (M) from an aqueous stream (A1) comprising vanillin or derivatives thereof. This step is a step of separating a solid phase: the biomass, from a liquid phase. The liquid phase obtained on conclusion of the biomass separation step is an aqueous phase (A1) comprising vanillin or derivatives thereof.

According to one aspect, the biomass separation step may be performed by filtration such as frontal filtration or tangential filtration, in particular membrane filtration, such as microfiltration, ultrafiltration, nanofiltration, or reverse osmosis. Membrane filtration may be performed by concentration or diafiltration. Advantageously, when the biomass separation step is frontal filtration, adjuvants may be added so as to improve the filtration efficacy.

In order to improve the solid/liquid separation efficacy, it is possible to perform several solid/liquid separation steps, notably to remove the smallest solid particles.

According to a particular aspect, the biomass separation step may be performed with one or more microfiltration membranes, in particular having a cut-off limit of about 0.2 μm, followed by one or more ultrafiltrations having a cut-off limit smaller than that of the microfiltration. In this configuration, the vanillin or derivatives thereof are dissolved in the aqueous phase and the biomass is retained by the membranes. Advantageously, ultrafiltration also allows the separation of molecules dissolved in the fermentation must.

In order to improve the yield of vanillin or derivatives thereof in the aqueous phase, a solvent, preferably water, is added during the filtration step. In general, the amount of solvent added is between 0.5 and 5 volume equivalents of fermentation must.

According to one aspect, microfiltration, ultrafiltration or diafiltration may be coupled with a nanofiltration or reverse osmosis step. Nanofiltration allows the concentration of vanillin in the retentate of the nanofiltration step to be increased, while the water passes through the membrane (permeate of the nanofiltration step). The permeate from this nanofiltration step may advantageously be recycled. In general, reverse osmosis has a cut-off limit of less than or equal to 100 Da. In general, nanofiltration has a cut-off limit of less than or equal to 400 Da. In general, nanofiltration has a cut-off limit of greater than or equal to 100 Da, for example a cut-off limit of between 100 and 250 Da.

Step (a0)

Prior to this step of separating the biomass from the aqueous solution of vanillin or derivatives thereof, the fermentation must may be stabilized. Thus, the process of the present invention may optionally comprise a step (a0) of stabilizing the fermentation must (M). Step (a) of the process according to the present invention may be performed on a stabilized fermentation must (M).

In the context of the present invention, the term “stabilization” refers to any method for preventing the degradation, notably by reduction, of vanillin or derivatives thereof between the end of fermentation and the purification process.

According to a first aspect, stabilization may be performed by the addition of at least one compound. The compound is preferentially chosen from sodium benzoate, ascorbic acid and salts thereof, potassium, calcium or sodium sorbate, zinc sulfate, propanoic acid, acetic acid or salts thereof, or sodium diacetate. Preferably, the amount of compound added is between 0.2 g/L and 6 g/L.

According to another aspect, stabilization may be performed by changing the temperature of the fermentation must. In general, the temperature is controlled so as to achieve a temperature of between 15° C. and 23° C., preferably between 18° C. and 21° C.

According to another aspect, stabilization may be performed by changing the pH of the fermentation must. In general, the pH is controlled so as to achieve a pH of less than or equal to 7.5, preferably less than or equal to 7, very preferentially less than or equal to 6.8. In general, the pH is controlled to achieve a pH of greater than or equal to 5.0, preferably greater than or equal to 6.

According to another aspect, the fermentation must may also be pasteurized. In general, the fermentation must is then heated to a temperature of between 50° C. and 90° C., preferably between 60° C. and 80° C. Heating is generally maintained for between 10 min and 120 min, preferably between 15 min and 45 min, for example for 20 min.

According to another aspect, the fermentation must may be stabilized by ultrasound. In general, ultrasound is emitted into the fermentation must for a period of between 10 min and 120 min.

According to a particular aspect, these aspects may be performed separately or in combination; thus, by way of nonlimiting example, it is possible to modify the temperature, the pH and to add a compound under the conditions described above.

Among these methods, methods without adding compounds are advantageous: without wishing to be bound by any theory, these methods allow possible deleterious effects on vanillin or derivatives thereof, notably in terms of odor or color, to be avoided.

However, the addition of certain compounds chosen for their compatibility with vanillin or derivatives thereof and the absence of side effects on vanillin or derivatives thereof may prove particularly advantageous in facilitating the subsequent process for purifying vanillin or derivatives thereof.

Step (b)

The purification process generally comprises at least one step of liquid/liquid extraction of the aqueous stream (A1), allowing the separation of an aqueous stream (A2) and an organic stream (O1) comprising vanillin or derivatives thereof. The liquid/liquid extraction step advantageously allows the vanillin or derivatives thereof in aqueous solution in the stream (A1) to be transferred into an organic phase (O1). Thus, on conclusion of the liquid/liquid extraction step, an organic phase (O1) comprising vanillin or derivatives thereof is obtained. The liquid/liquid extraction step advantageously allows the separation of vanillin or derivatives thereof from water-soluble compounds such as vanillic acid or ferulic acid in salified or protonated form.

In general, the pH of the aqueous solution is between 5 and 8, preferably between 5.5 and 7.5, very preferentially between 6 and 7.

In general, the extraction solvent is chosen for its capacity for dissolving vanillin or derivatives thereof; advantageously, the solvent may be of biobased origin. According to one aspect, the chosen solvent is compatible with food industry standards, notably FEMA GRAS, and is water-immiscible. Preferably, the solvent is chosen from methyl ethyl ketone, methyl isobutyl ketone, ethyl acetate, isopropyl acetate, cyclohexane or mixtures thereof.

The liquid/liquid extraction step may be a discontinuous liquid/liquid extraction. In order to maximize the amount of vanillin or derivatives thereof obtained in the organic phase (O1), the volume ratio of solvent relative to the aqueous solution (A1) of vanillin or derivatives thereof is between 1:5 and 5:1, preferably between 1:1 and 5:1, preferably between 1.5:1 and 3:1.

According to another aspect, the liquid/liquid extraction step may be performed continuously. In general, the volume ratio of solvent relative to the aqueous solution (A1) of vanillin or derivatives thereof is between 5:1 and 1:2, preferably between 3:1 and 1:1.

The yield of vanillin or derivatives thereof from the liquid/liquid extraction step is generally greater than or equal to 95%, preferably greater than or equal to 97%, very preferentially greater than or equal to 98%.

According to a particular aspect, the biomass separation and liquid/liquid extraction steps may be performed simultaneously. This particular aspect allows vanillin or derivatives thereof to be extracted into the organic solvent (O1) from the fermentation must (M). The biomass is separated from the two-phase system. This process is advantageous in that the loss of vanillin or derivatives thereof in the biomass is reduced. This separation step may notably be performed continuously by centrifugation, in particular using a two-phase or three-phase centrifuge or a countercurrent extractor.

The organic stream (O1) comprising vanillin or derivatives thereof obtained on conclusion of the liquid/liquid extraction step generally has a concentration of vanillin or derivatives thereof of between 0.1% by weight and 10% by weight. The organic stream (O1) also includes the solvent used during the liquid/liquid extraction. The stream (O1) may also comprise other compounds such as ferulic acid and derivatives thereof, vanillic acid, vanillyl alcohol or derivatives thereof, coumaric acid, para-hydroxybenzaldehyde, or guaiacol.

Optionally, a stripping step may be performed on the aqueous stream (A2) obtained on conclusion of the liquid/liquid extraction step. Advantageously, the stripping step allows the recovery of organic compounds contained in the aqueous stream (A2), notably vanillin or derivatives thereof. Thus, stripping enables the yield of vanillin or derivatives thereof to be improved. The stripping step also facilitates the treatment of the aqueous effluents.

Step (c)

The process of the present invention comprises at least one step (c) of concentrating an organic stream (O1) to obtain an organic stream (O2) comprising vanillin or derivatives thereof, in which compounds with a boiling point lower than that of vanillin or derivatives thereof are separated from said organic stream (O1). In particular, the concentrating step (c) allows the solvent used in the liquid/liquid extraction to be evaporated.

In general, on conclusion of the concentrating step (c), vanillin or derivatives thereof are in liquid or solid form, depending on the temperature. In general, at least 95% of the solvent has been separated from the vanillin or derivatives thereof. However, the concentrating step may consist of one or more concentrating steps.

Thus, according to a particular aspect, at least 95% of the amount of solvent present in the organic stream (O1) of vanillin may be evaporated.

According to another aspect, the solvent contained in the vanillin organic stream (O1) is partially evaporated to obtain an organic stream (O2).

Preferably, the vanillin concentration in the organic stream (O2) is higher than the vanillin concentration in the organic stream (O1).

According to a particular aspect, the organic stream (O1) of vanillin or derivatives thereof is concentrated so as to obtain an organic stream (O2) of vanillin with a concentration of between 10% by weight and 95% by weight.

The organic stream (O2) may be subjected to one or more concentrating steps so as to obtain vanillin or derivatives thereof in liquid or solid form as a function of the temperature.

The concentrating step may be performed in a continuous or batch process.

In a batch process, the concentrating step may be performed in a stirred reactor or in a distillation column. Preferentially, the concentrating step is performed under vacuum, preferably at a pressure of between 5 mbar and 300 mbar, more preferably between 50 mbar and 250 mbar. The concentrating step is generally performed at a temperature of between 25° C. and 100° C.

In a continuous process, the concentrating step may be performed using tray or packed distillation columns, falling-film evaporators, scraped-film evaporators or baffle columns.

In general, the concentrating step is performed at a distillation head pressure of between 0.5 bar and 5 bar, preferably between 1 bar and 3 bar. The temperature at the distillation head is generally between 75° C. and 150° C., preferably between 85° C. and 120° C. and very preferably between 90° C. and 110° C.

Advantageously, the concentrating step is performed so as to reduce the residence time of vanillin or derivatives thereof in the reactors or columns.

The concentrating step allows the recovery of the organic stream (O2) comprising vanillin or derivatives thereof at the bottom of the column, while the solvent and, more generally, compounds with boiling points lower than that of vanillin or derivatives thereof are recovered at the top of the column. The organic stream (O2) comprising vanillin or derivatives thereof generally has a purity of greater than or equal to 90%. Other compounds may be present in the organic stream (O2), notably vanillyl alcohol or derivatives thereof, vanillic acid, acetovanillone, 4-((4-hydroxy-3-methoxybenzyl)oxy)-3-methoxybenzaldehyde and 4-hydroxy-3-(4-hydroxy-3-methoxybenzyl)-5-methoxybenzaldehyde.

Step (d)

The process of the present invention may comprise at least one step in which compounds with a boiling point higher than that of vanillin or derivatives thereof are separated from an organic stream (O2) comprising vanillin or derivatives thereof to obtain a stream (O3) comprising vanillin or derivatives thereof. This step may be a de-tarring step.

Step (d) may be performed in a distillation column, a baffle column, a falling-film evaporator, or a scraped-film evaporator.

According to a particular embodiment, step (d) may be performed in the presence of a technical adjuvant, for example a fluidizer. Thus, during step (d) or prior to step (d), a fluidizer, preferably one permitted by the regulations for food products, for example polyethylene glycol, may be added.

Step (e)

The process of the present invention includes at least one step of crystallizing vanillin or derivatives thereof contained in the stream (O3).

The crystallization step allows the purification of a stream (O3) comprising vanillin or derivatives thereof. Preferably, the stream (O3) has a purity of between 85% and 99%, preferably greater than or equal to 90%, very preferentially greater than or equal to 95%. Advantageously, the crystallization step allows the production of purified vanillin or derivatives thereof with a purity of greater than or equal to 95%, preferably greater than or equal to 97%, very preferentially greater than or equal to 99%. Vanillin or derivatives thereof with a purity of between 85% and 99% may also comprise at least one other compound chosen from vanillyl alcohol or derivatives thereof, vanillic acid, guaiacol, acetovanillone, 4-((4-hydroxy-3-methoxybenzyl)oxy)-3-methoxybenzaldehyde and 4-hydroxy-3-(4-hydroxy-3-methoxybenzyl)-5-methoxybenzaldehyde.

The crystallization is generally performed in an alcoholic solution. Preferably, the solvent used for the crystallization may be a water-soluble alcohol, preferably ethanol. The solvent used for the crystallization may be a water/alcohol mixture. In general, the amount of alcohol is between 2% and 40% by mass, preferably between 5% and 35% by mass, and very preferentially between 15% and 25% by mass. In general, during crystallization, the vanillin concentration at the start of crystallization is between 5% and 60% by weight, preferably between 10% and 50% by weight, advantageously between 15% and 35% by weight, and even more preferentially between 15% and 25%. Very advantageously, crystallization allows the separation of vanillin or derivatives thereof from the impurities contained in the stream (O3), and this separation is advantageously performed without degradation of the vanillin or derivatives thereof. Crystallization takes place at a temperature of between 0° C. and 50° C. The crystallization yield is generally greater than or equal to 80%.

The vanillin or derivatives thereof obtained on conclusion of the crystallization step generally have a color in ethanolic solution at 10% by weight of less than or equal to 150 Hazen, preferably less than or equal to 100 Hazen, and very preferentially less than or equal to 50 Hazen. Moreover, the vanillin or derivatives thereof obtained on conclusion of the purification process according to the present invention have compliant organoleptic properties.

### EXAMPLE

3 kg of a stabilized fermentation must as defined in Table 1 are subjected to a purification process as described hereinbelow.

TABLE 1
Composition of the fermentation must

| | | |
|---|---|---|
| Biomass | 1.5% | by weight |
| Vanillin | 2% | by weight |
| Water | 95% | by weight |
| Other compounds | 1.5% | by weight |

After separating the biomass from the aqueous phase by centrifugation, the vanillin is extracted with 6 kg of ethyl acetate.

The solvent in the upper phase is totally distilled off using a rotavapor at 50° C. under a vacuum of 100 mbar, to obtain a medium with a vanillin concentration of 78%.

This vanillin-concentrated medium is finally purified according to the following two steps:
removal of the heavy products (compounds less volatile than vanillin) by distillation on a scraped-film evaporator at 155° C. and 5 mbar, to obtain a vanillin concentration >90%,
the latter medium is crystallized by dissolving it in a stirred reactor containing a water/ethanol mixture (9/2) at 40° C. to obtain a vanillin concentration of 20%. The whole system is cooled to 0° C. with stirring. The crystallized vanillin is filtered off on a Büchner filter, washed with cold water and dried in an oven at 50° C. under 200 mbar for 20 hours.

The purity of the vanillin obtained is 99.7%, with a color in ethanolic solution at 10% by weight of 19 Hazen. The crystallized vanillin also has good organoleptic properties.

1. A process for purifying a fermentation must (M), obtained via a biotechnological process, comprising biomass and vanillin or derivatives thereof, said purification process comprising:
(a) a step of separating the biomass of a fermentation must (M) from an aqueous stream (A1) comprising vanillin or derivatives thereof,
(b) at least one step of liquid/liquid extraction of the aqueous stream (A1) comprising vanillin or derivatives thereof, said extraction step allowing the separation of an aqueous stream (A2) and an organic stream (O1) comprising vanillin or derivatives thereof,
(c) at least one step of concentrating the organic stream (O1) to obtain an organic stream (O2) in which compounds with a boiling point lower than that of vanillin or derivatives thereof are separated from said organic stream (O1),
(d) at least one step in which compounds with a boiling point higher than that of vanillin or derivatives thereof are separated from an organic stream (O2) comprising vanillin or derivatives thereof to obtain a stream (O3),
(e) at least one step of crystallizing the vanillin or derivatives thereof contained in the stream (O3).

2. The process of claim 1, characterized in that steps (a) and (b) are performed simultaneously.

3. The process of claim 1, further comprising a step (a0) prior to step (a), of stabilizing the fermentation must (M).

4. The process of claim 1, in which step (a) is performed by filtration.

5. The process of claim 1, in which the extraction solvent of step (b) is chosen from methyl ethyl ketone, methyl isobutyl ketone, ethyl acetate, isopropyl acetate, cyclohexane or mixtures thereof.

6. The process of claim 1, in which step (c) is performed with tray or packed distillation columns, falling-film evaporators, scraped-film evaporators or baffle columns.

7. The process of claim 1, in which step (d) is performed in a distillation column, a baffle column, a falling-film evaporator, or a scraped-film evaporator.

8. The process of claim 1, in which the vanillin or derivatives thereof obtained on conclusion of the crystallization step have a color in ethanolic solution at 10% by weight of less than or equal to 150 Hazen.

9. The process of claim 1, in which the vanillin or derivatives thereof obtained on conclusion of the crystallization step have a color in ethanolic solution at 10% by weight of less than or equal to 100 Hazen.

10. The process of claim, in which the vanillin or derivatives thereof obtained on conclusion of the crystallization step have a color in ethanolic solution at 10% by weight of less than or equal to 50 Hazen.

11. The process of claim 4, in which the filtration is frontal filtration or tangential filtration

12. The process of claim 4, in which the filtration is membrane filtration.

13. The process of claim 12, in which the membrane filtration is microfiltration, ultrafiltration, nanofiltration, or reverse osmosis.
